(19) United States
(12) Patent Application Publication
(10) Pub. No.: US 2024/0289707 A1
(43) Pub. Date: Aug. 29, 2024

(54) METHOD AND SYSTEM TO TRACK AND OPTIMIZE VEHICLE OPERATORS PERFORMANCE

(71) Applicant: Mirza Muhammad Ajmal Beg, Calgary (CA)

(72) Inventor: Mirza Muhammad Ajmal Beg, Calgary (CA)

(21) Appl. No.: 18/584,944

(22) Filed: Feb. 22, 2024

Related U.S. Application Data
(60) Provisional application No. 63/486,994, filed on Feb. 26, 2023.

(51) Int. Cl. G06Q 10/0631; G06Q 10/047; G16H 50/30

(52) U.S. Cl. CPC G06Q 10/063112; G06Q 10/047; G16H 50/30

(57) ABSTRACT

The vehicle operators performance tracking and monitoring system assigns trips to the vehicle operators while considering anticipated physical and mental health of the vehicle operators which can impact performances of vehicle operators during the assigned trips. The system also considers performance impacting factors associated with segments of routes of trips and ability of the vehicle operators to reduce impact of the extent performance impacting factors while assigning the trips to the vehicle operators. The system further considers maintenance status of the vehicles while assigning the trips to the vehicle operators. The system also checks compliance with the Occupational Health & Safety laws/rules/regulations during the assignment of the trips and allows auto adjustment of the trip data to remove performance impacting factors which are out of control of vehicle operators to build accurate models. Multiple organizations share the vehicle operators performance tracking and monitoring system.

### CROSS REFERENCE TO RELATED APPLICATIONS

This application claims priority of U.S. Provisional Application 63/486,994 (METHOD AND SYSTEM TO TRACK AND OPTIMIZE VEHICLE OPERATOR PERFORMANCE) filed Feb. 26, 2023 which is further based on U.S. 63/314,426 (METHOD AND SYSTEM TO TRACK AND OPTIMIZE VEHICLE OPERATOR PERFORMANCE) filed on Feb. 27, 2022. U.S. Provisional Application 63/486,994 is incorporated herein by reference.

### BACKGROUND OF THE INVENTION

### Field of the Invention

The present invention is directed to a system, computer-implemented method and storage medium with computer executable instructions to track and optimize vehicle operators performance.

### Description of the Related Art

The current vehicle operators performance tracking and monitoring systems are not designed to fully optimize the performance of the vehicle operator performance. The current vehicle operators performance tracking and monitoring systems do not support tracking of multiple types of vehicle operators (such as passenger ferry operator and passenger bus operator) within the same system, thus requiring extra coordination and effort. The current vehicle operators performance tracking and monitoring systems further do not assign trips to the vehicle operators while considering anticipated physical and mental health of the vehicle operators which can impact performances of vehicle operators during the trips. The current vehicle operators performance tracking and monitoring systems further do not consider performance impacting factors associated with segments of routes of trips and ability of the vehicle operators to reduce impact of the extent performance impacting factors while assigning the trips to the vehicle operators. The current vehicle operators performance tracking and monitoring systems further do not take into account maintenance status of the vehicles. The current vehicle operators performance tracking and monitoring systems further check compliance with the health and safety laws during the assignments of the trips. The current vehicle operators performance tracking and monitoring systems further do not allow auto adjustment of data to remove performance impacting factors which are out of control of vehicle operators to build accurate performance models.

### SUMMARY OF THE INVENTION

In one embodiment of the present invention, the vehicle operators performance tracking and monitoring system assigns trips to the vehicle operators while considering anticipated physical and mental health of the vehicle operators which can impact performances of vehicle operators during the assigned trips. In another embodiment of the present invention, the vehicle operators performance tracking and monitoring system considers performance impacting factors associated with segments of routes of trips and ability of the vehicle operators to reduce impact of the extent performance impacting factors while assigning the trips to the vehicle operators. In another embodiment of the present invention, the vehicle operators performance tracking and monitoring system considers maintenance status of the vehicles while assigning the trips to the vehicle operators. In another embodiment of the present invention, the vehicle operators performance tracking and monitoring system checks compliance with the Occupational Health & Safety laws/rules/regulations during the assignment of the trips. In another embodiment of the present invention, the vehicle operators performance tracking and monitoring system allows auto adjustment of the trip data to remove performance impacting factors which are out of control of vehicle operators to build accurate models. In another embodiment, multiple organizations share the vehicle operators performance tracking and monitoring system.

### BRIEF DESCRIPTION OF THE DRAWINGS

FIG. 1 shows a block diagram of an example computer used to provide computing functionalities to implement the present invention.

FIG. 2 conceptually illustrates an exemplary vehicle operators performance tracking and monitoring system according to the present invention.

FIG. 3 is a flow chart illustrating an exemplary operation according to the present invention.

### DETAILED DESCRIPTION OF THE INVENTION

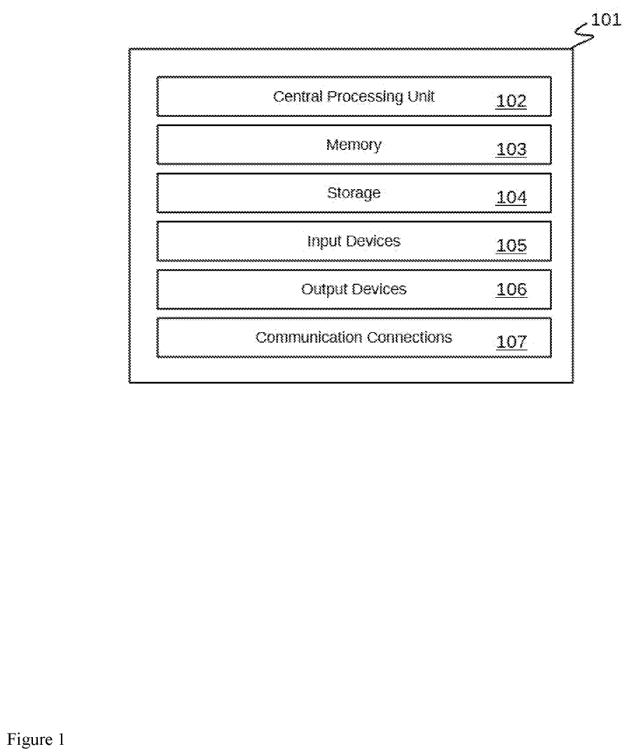

FIG. 1 shows a block diagram of an example computer 101 used to provide computing functionalities to implement the present invention. Computing devices such as laptop, workstation, laptops, distributed computing systems, desktop, server, cluster, virtual machine, mainframe, smart phone, wireless data port, a personal digital assistance, tablet computing devices are examples of such computer 101. The computer 101 includes a Processor 102 (e.g., a central processing unit (CPU), microprocessor, a digital signal processor (DSP), a conventional processor, a virtual process, micro-controller, virtual machine, a graphic processing unit (GPU), a radio-frequency integrated circuit (RFIC), an application specific integrated circuit (ASIC) or any suitable combination thereof). Processor 102 can be a multi-core processor or a plurality of multi-core processors. Memory 103 can be any kind of one or more memory devices such as read only memory (ROM), random access memory (RAM), optical, magnetic and flash memory. In some implementation, Memory 103 can be combination of two or more different types of memory. Memory 103 is shown as integral part of the computer 101, in alternative implementation memory 103 can be external to computer 101. Storage device 104 can be any medium which can be used as persistence storage. For example, hard drive, tape drive, optical disk drive, USB, flash driver and disk arrays. The storage device 104 is not limited to a particular storage device and may include memory devices such as ROM, RAM, hard disk, and the like. The storage device 104 may be a cloud storage device. Input device 105 is used to input external data and can be any kind of device such as mouse, trackball, light pen, bio-metric mechanism including voice recognition. Output device 106 can be any kind of device used for data output. For example, cathode-ray-tube (CRT) monitor, plasma display, crystal display, projector, printer and speaker. Communication interface 107 can be one or more interfaces to any kind of networks such as internet, intranet, local area network, wide area network, a telephone network such as Public Switched Telephone Network, or combination of different kinds of networks. In some exemplary embodiments, the storage device 104 may connect to the example computer 101 using communication interface 107. The example computer system 101 can be a virtual machine or a cloud based computing device. The expression “or” is not exclusive throughout the specification.

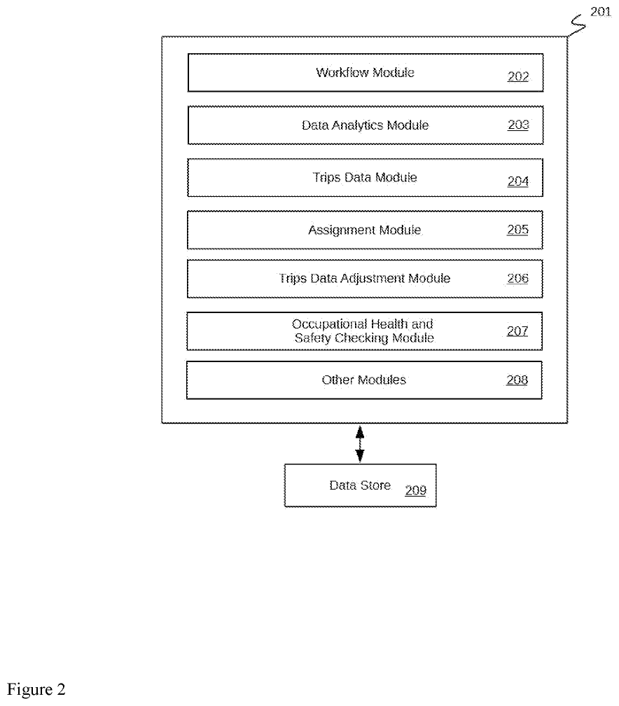

FIG. 2 is a diagram conceptually illustrating an exemplary vehicle operators performance tracking and monitoring system 201 according to the present invention. The exemplary vehicle operators performance tracking and monitoring system 201 stores data in data store 209. The data store 209 may also include one or more data schema, one or more constraints, one or more default values, one or more validations, one or more default values, one or more data types information, one or more format information, one or more data transformation information, one or more data extraction information, one or more presentation templates, one or more filters, one or more insights, one or more recommendations, one or more predictions, one or more events and other configuration data which is used to create, read, delete and update the stored data in the data store 209. The stored rules in the data store 209 may be based on one or more self-learning algorithms. The data store 209 can be one or more distributed data stores. The data store 209 can be a partially or fully one or more cloud based. The data store 209 may be accessible through one or more interfaces. The data store 209 may store data as structured data and/or as non-structured data. The exemplary vehicle operators performance tracking and monitoring system 201 generally consists of multiple modules 202-208. It is a known art to a person skilled in the art that the modules can be split, merged and renamed. All such variations are within scope of the invention. It is also a known art to a person skilled in the art that a ‘module’ can be replaced with other means of grouping relevant computer instructions such as in form of function, subroutine, routine, component, templates, class, business objects, services and micro-services. All such variations known to a person skilled in the art are within the scope of the present invention. It is also a known art that modules can be distributed on multiple computing environments. Such variations known to a person skilled in the art are within the scope of the present inventions.

The Workflow Module 202 contains computer instructions to manage and execute workflows within the exemplary vehicle operators performance tracking and monitoring system 201. The Data Analytics Module 203 contains computer instructions to manage and execute steps to perform data analytics within the exemplary vehicle operators performance tracking and monitoring system 201. The Trips Data Module 204 contains computer instructions to manage and execute steps related to the trips data within the exemplary vehicle operators performance tracking and monitoring system 201. The Assignment Module 205 contains computer instructions to manage and execute steps related to assigning trips, routes, vehicles and vehicle operators for the exemplary vehicle operators performance tracking and monitoring system 201. The Trips Data Adjustment Module 206 contains computer instructions to manage and execute steps related to adjustment of the trip data with or without human input within the exemplary vehicle operators performance tracking and monitoring system 201. The Occupational Health and Safety Checking Module 207 contains computer instructions to manage and execute steps related to Occupational Health and Safety checks within the exemplary vehicle operators performance tracking and monitoring system 201. The Other Modules 208 contains computer instructions to execute steps not performed by the Modules 202-207 such as:
Managing definitions of organizations
Managing definitions of vehicle types
Managing definitions of fleet types
Managing definitions of target market segments
Managing definitions of vehicle operator groups
Managing definitions of trip types
Managing definitions of geographic regions
Managing definitions of vehicle models
Managing access controls
Managing system user subscription
Managing data growth controls
Managing system security
Managing users
Managing interfaces
Managing system fail-overs configurations

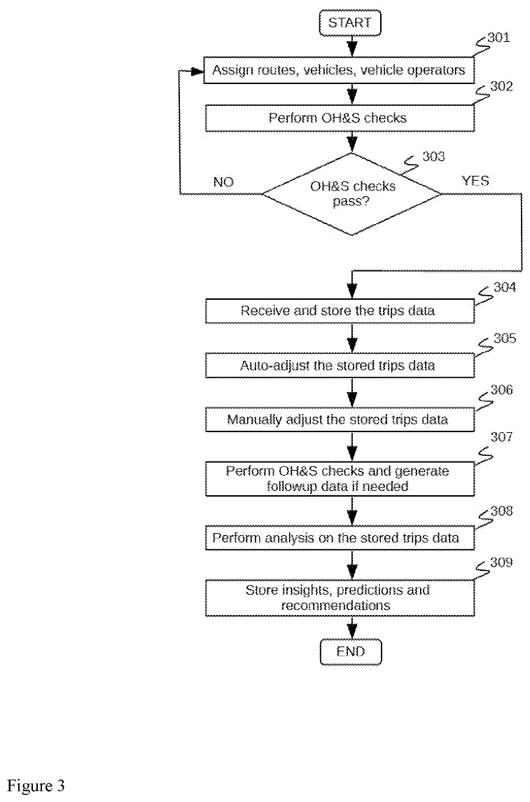

FIG. 3 is a flow chart illustrating an exemplary operation according to the present invention.
Step 301 is implemented by the Assignment Module 205 within the vehicle operators performance tracking and monitoring system 201 optimizes one or more vehicle operators performance by assigning one or more segments of one or more routes and one or more vehicles to the one or more vehicle operators for one or more trips based one or more of following (a), (b) and data (c):
(a) anticipated physical and mental health of the one or more vehicle operators while operating the one or more vehicles during the one or more trips,
(b) extent of one or more performance impacting factors associated with the one or more segments of the one or more routes and ability of the one or more vehicle operators to reduce impact of the extent of the one or more performance impacting factors during the one or more trips.
(c) stored one or more maintenance histories associated with the one or more vehicles.
The expression “vehicle” may represent a water vehicle such as passenger ferry, or a flying vehicle such as passenger plane, or a land vehicle such as a train/a garbage collection truck. The expression “vehicle” may also represent a vehicle which is both land vehicle and water vehicle at the same time such as hovercraft. The expression “vehicle” may also represent a vehicle which is both land vehicle and air vehicle such as flying taxi. The expression “vehicle” may also represent a vehicle which is both air vehicle and water vehicle such as sea plane. The expression “vehicle operator” may represent a vehicle operator who operates a vehicle. The “vehicle operator” may be assisted by one or more auto-operation functions such as car cruising function. The vehicle may be operated by one or more vehicle operators. The expression “route” represents one or more paths along/on which the vehicle is operated. The expression “route” may be represented by one or more segments. One or more segments may overlap with one another. One or more segments may be defined by using one or more specific attributes and one or more associated values such as: segment ABC of route DEF with traffic congestion level of HIGH. The expression “trip” may represent movement of one or more vehicles along one or more routes by one or more vehicle operators. The expression “anticipated physical and mental health” is one or more attributes and associated one or more values calculated by the computer with or without human input and which represents expected physical and/or mental health condition of one or more vehicle operators while operating one or more vehicles along/on one or more segments of one or more routes during the one or more trips. The extent of one or more performance impacting factors may be represented by one or more numerical and/or one or more literal values such as high, very high, low and very low. The ability of the one or more vehicle operators to reduce impact of the extent of the one or more performance impacting factors during the one or more trips, is represented by one or more attributes and associated one or more values calculated by the computer with or without human input. The impact of the extent of the one or more performance impacting factors may be represented by one or more numerical and/or one or more literal values such as high, very high, low and very low. The expression “stored” refers to information stored in the data store 201.

The Assignment Module 205 may determining the anticipated physical and mental health based on one or more stored weight profiles and one or more of the following:
Stored historical data related to slept hours associated with the one or more vehicle operators.
Stored historical data related to time being away from home associated with the one or more vehicle operators.
Stored historical data related to sitting down over one or more specific limits in one or more vehicles associated with the one or more vehicle operators.
Stored historical data related to one or more wait times over one or more specific limits at one or more border crossings associated with the one or more vehicle operators.
Stored historical data related to histories of use of one or more rest areas associated with the one or more vehicle operators.
Stored historical data related to encountered one or more incidents of harassment during one or more duties associated with the one or more vehicle operators.
Stored historical data in the data store 209 related to one or more physical and mental disabilities associated with the one or more vehicle operators.
Stored historical data in the data store 209 related to encountered one or more incidents of abuse during duties associated with the one or more vehicle operators.
Stored historical data in the data store 209 related to one or more food intake breaks associated with the one or more vehicle operators.
Stored historical data in the data store 209 related to one or more observations of one or more traffic accidents associated with the one or more vehicle operators.
Stored historical data in the data store 209 related to one or more specific types of cargo associated with the one or more vehicle operators.
Stored historical data in the data store 209 related to one or more completion wait times for one or more procedural processing above one or more specific thresholds associated with the one or more vehicle operators.
Stored historical data in the data store 209 related to one or more travels through unsafe one or more areas associated with the one or more vehicle operators.
Stored historical data in the data store 209 related to experienced one or more traffic jams with one or more specific characteristics associated with the one or more vehicle operators.
Stored historical data in the data store 209 related to one or more personal traumas associated with the one or more vehicle operators.

A weight may indicate relevance of the above stored historical data while calculate the anticipated physical and mental health and may be represented by one or more numerical and/or one or more literal values. The one or more weight profiles may be further specific to one or more of the following:
one or more organizations
one or more fleet types
one or more target market segments
one or more vehicle operator groups
one or more trip types
one or more geographic regions
one or more vehicle models

The expression “organization” may also represent an organizational unit within one or more organizations. The organization may be in hierarchical relationship with another organization. The organization may be a partially or fully logical organization rather than a physical organization.

The one or more performance impacting factors associated with the one or more segments of the one or more routes may include one or more of the following:
One or more weather conditions such as foggy weather.
One or more traffic conditions such as morning rush hour or large number of landing flights during a specific holiday season.
One or more vehicle speed conditions such as highway without speed limits.
One or more route conditions such as bad road patching condition and pit holes.
One or more parking conditions such as tight parking spots.
One or more load weight conditions such as carrying heavy load.
One or more load type conditions such as transporting fragile load.
One or more stopping conditions such as hard to land runway.

The extent of the one or more performance impacting factors associated with the one or more segments of the one or more routes may be represented as stored temporal data. The ability of the one or more vehicle operators to reduce impact of the extent of the one or more performance impacting factors may be represented as stored temporal data.

The extent of one or more performance impacting factors associated with the one or more segments of the one or more routes may be determined by the Assignment Module 205 based one or more of the following:
Stored historical data related to the extent of one or more performance impacting factors associated with the one or more segments of the one or more routes.
One or more stored insights based on the stored historical data related to the extent of one or more performance impacting factors associated with the one or more segments of the one or more routes.
One or more stored configurations.
One or more stored rules based on the stored historical data related to the extent of one or more performance impacting factors associated with the one or more segments of the one or more routes.
One or more stored models based on the stored historical data related to the extent of one or more performance impacting factors associated with the one or more segments of the one or more routes.
One or more inputs provided through one or more graphical user interfaces.
One or more inputs provided without one or more graphical user interfaces.

The ability of the one or more vehicle operators to reduce impact of the extent of the one or more performance impacting factors may be determined by the Assignment Module 205 based one or more of the following:
Stored historical data related to the ability of the one or more vehicle operators to reduce impact of the extent of the one or more performance impacting factors.
One or more stored insights based on the stored historical data related to the ability of the one or more vehicle operators to reduce impact of the extent of the one or more performance impacting factors.
One or more stored configurations.
One or more stored rules based on the stored historical data related to the ability of the one or more vehicle operators to reduce impact of the extent of the one or more performance impacting factors.
One or more stored models based on the stored historical data related to the ability of the one or more vehicle operators to reduce impact of the extent of the one or more performance impacting factors.
One or more inputs provided through one or more graphical user interfaces.
One or more inputs provided without one or more graphical user interfaces.
   The anticipated physical and mental health of the one or more vehicle operators while operating the one or more vehicles is determined based one or more of the following:
Stored historical data related to the physical and mental health of the one or more vehicle operators while operating the one or more vehicles.
One or more stored insights based on the stored historical data related to the physical and mental health of the one or more vehicle operators while operating the one or more vehicles.
One or more stored configurations.
One or more stored rules based on the stored historical data related to the physical and mental health of the one or more vehicle operators while operating the one or more vehicles.
One or more stored models based on the stored historical data related to the physical and mental health of the one or more vehicle operators while operating the one or more vehicles.
One or more inputs provided through one or more graphical user interfaces.
One or more inputs provided without one or more graphical user interfaces.

Step 302 implemented by the Occupational Health & Safety Module 205 within the vehicle operators performance tracking and monitoring system 201 checks that the assigned one or more segments of the one or more routes and the one or more vehicles to the one or more vehicle operators for the one or more trips comply with the occupational health and safety rules/laws/regulations using one or more rules related to the occupational health and safety rules/laws/regulations stored in the data store 209. Step 302 implemented by the Occupational Health & Safety Module 205 within the vehicle operators performance tracking and monitoring system 201, verifies result of the checks. In another exemplary embodiment, Step 301 may be executed in parallel to Step 302.

Step 304 implemented by the Trips Data Module 204 within the vehicle operators performance tracking and monitoring system 201 receives and stores data related to the one or more trips assigned at Step 301 that the one or more vehicle operators have partially or fully completed. The received trips data may be received in one or more batches and/or as one or more data streams. The received trips data may be received in different formats through one or more communication protocols using one or more communication channels. The received data may go through one or more extract transform load (ETL) and/or extract load transform (ELT) processes

Step 305 implemented by the Trips Data Adjustment Module 206 auto-adjusts the data stored at Step 304. The act of auto-adjusting the data here may also refer to generating additional relevant data with or without changing the data stored at Step 304. The Trips Data Adjustment Module may require additional data (such as weather along a specific trip route at a certain date and time) to auto-adjust the stored data. The required additional data may be received from one or more external and/or one or more internal data sources. One or more extents of one or more auto-adjustments by the Trips Data Adjustment Module 206 may be further specific to one or more of the following:
one or more organizations
one or more fleet types
one or more target market segments
one or more vehicle operator groups
one or more trip types
one or more geographic regions
one or more vehicle models

Step 306 implemented by the Trips Data Adjustment Module 206 allows one or more manual adjustments for the data stored at Step 304 and/or data which is auto-adjusted at Step 305. The Trips Data Adjustment Module 206 and the Workflow Module 202 may initiate one or more workflows for the one or more manual adjustments when one or more stored predefined conditions are satisfied. In another exemplary embodiment, Step 306 may be executed before Step 305. In another exemplary embodiment, Step 305 and Step 306 may be executed in parallel.

Step 307 implemented by the Occupational Health & Safety Module 205 within the vehicle operators performance tracking and monitoring system 201 checks that the one or more trips associated the received trips data comply with the Occupational Health and Safety rules/laws/regulations using one or more rules related to the occupational health and safety rules/laws/regulations stored in the data store 209. The Occupational Health & Safety Module 205 within the vehicle operators performance tracking and monitoring system 201, generates followup data when needed. Step 307 is optional. In other exemplary embodiments, Step 305, Step 306 and Step 307 may perform checks related to the Occupational Health and Safety rules/laws/regulations.

Step 308 implemented by the Data Analytics Module 206 uses the stored trips data from one or more of Step 304, Step 305 and Step 306 to generates one or more of the following:
One or more insights.
One or more predictions.
One or more recommendations.

Step 309 implemented by the Data Analytics Module 206 stores the generated one or more insights, one or more predictions and one or more recommendations. It is a well known art that acts/steps in any flow chart can be split, merged and reordered. Such variations to acts/steps in FIG. 3 are within scope of the invention.

The Workflow Module 202 implements one or more workflows allowing interaction through one or more user graphical interfaces for managing one or more life cycles (such as creation, deleting and updating) of one or more of the following:
The one or more performance impacting factors.
The one or more anticipated physical and mental health data.

It is to be understood that while the detailed description describes the present invention, the foregoing description is for illustrative purpose and does not limit the scope of the present invention which is defined by the scope of the appended claims. Other embodiments, arrangements and equivalents will be evident to those skilled in the art. Such other embodiments, arrangements and equivalents are within the scope of the present invention as defined by the appended claims.

1. A system for tracking and optimizing one or more vehicle operators performance, the system comprising:
one or more processors coupled to one or more memories:
one or more computer-executable instructions configured to assign one or more segments of one or more routes and one or more vehicles to the one or more vehicle operators for one or more trips based on:
anticipated physical and mental health of the one or more vehicle operators while operating the one or more vehicles during the one or more trips;
extent of one or more performance impacting factors associated with the one or more segments of the one or more routes and ability of the one or more vehicle operators to reduce impact of the extent of the one or more performance impacting factors during the one or more trips.

2. The system of claim 1, further comprising one or more computer-executable instructions configured to determine the anticipated physical and mental health based on one or more stored weight profiles and one or more of the following:
historical data related to slept hours associated with the one or more vehicle operators;
historical data related to time being away from home associated with the one or more vehicle operators;
historical data related to sitting down over one or more specific limits in one or more vehicles associated with the one or more vehicle operators;
historical data related to one or more wait times over one or more specific limits at one or more border crossings associated with the one or more vehicle operators;
historical data related to histories of use of one or more rest areas associated with the one or more vehicle operators;
historical data related to encountered one or more incidents of harassment during one or more duties associated with the one or more vehicle operators;
historical data related to one or more physical and mental disabilities associated with the one or more vehicle operators;
historical data related to encountered one or more incidents of abuse during duties associated with the one or more vehicle operators;
historical data related to one or more food intake breaks associated with the one or more vehicle operators;
historical data related to one or more observations of one or more traffic accidents associated with the one or more vehicle operators;
historical data related to one or more specific types of cargo associated with the one or more vehicle operators;
historical data related to one or more completion wait times for one or more procedural processing above one or more specific thresholds associated with the one or more vehicle operators;
historical data related to one or more travels through unsafe one or more areas associated with the one or more vehicle operators;
historical data related to experienced one or more traffic jams with one or more specific characteristics associated with the one or more vehicle operators;
historical data related to one or more personal traumas associated with the one or more vehicle operators.

3. The system of claim 2, wherein the one or more weight profiles are further specific to one or more of the following:
one or more organizations;
one or more fleet types;
one or more target market segments;
one or more vehicle operator groups;
one or more trip types;
one or more geographic regions;
one or more vehicle models.

4. The system of claim 1, wherein the one or more performance impacting factors associated with the one or more segments of the one or more routes include one or more of the following:
one or more weather conditions;
one or more traffic conditions;
one or more speed conditions;
one or more route conditions;
one or more parking conditions;
one or more load weight conditions;
one or more load type conditions;
one or more stopping conditions.

5. The system of claim 1, further comprising one or more computer-executable instructions configured to assign the one or more segments of the one or more routes and the one or more vehicles to the one or more vehicle operators further based on stored one or more maintenance histories associated with the one or more vehicles.

6. The system of claim 1, wherein the extent of the one or more performance impacting factors associated with the one or more segments of the one or more routes is temporal.

7. The system of claim 1, wherein the ability of the one or more vehicle operators to reduce the impact of the extent of the one or more performance impacting factors is temporal.

8. The system of claim 1, further comprising one or more computer-executable instructions configured to determine the extent of the one or more performance impacting factors associated with the one or more segments of the one or more routes based on stored historical data related to the extent of the one or more performance impacting factors associated with the one or more segments of the one or more routes and further optionally based on one or more of the following:
one or more stored insights based on the stored historical data related to the extent of the one or more performance impacting factors associated with the one or more segments of the one or more routes;
one or more stored rules based on the stored historical data related to the extent of one or more performance impacting factors associated with the one or more segments of the one or more routes wherein the one or more stored rules are further optionally based on one or more self-learning algorithms;
one or more stored models based on the stored historical data related to the extent of one or more performance impacting factors associated with the one or more segments of the one or more routes.

9. The system of claim 1, further comprising one or more computer-executable instructions configured to determine ability of the one or more vehicle operators to reduce the impact of the extent of the one or more performance impacting factors based on stored historical data related to the ability of the one or more vehicle operators to reduce impact of the extent of the one or more performance impacting factors and further optionally based on one or more of the following:
one or more stored insights based on the stored historical data related to the ability of the one or more vehicle operators to reduce impact of the extent of the one or more performance impacting factors;
one or more stored rules based on the stored historical data related to the ability of the one or more vehicle operators to reduce impact of the extent of the one or more performance impacting factors wherein the one or more stored rules are further optionally based on one or more self-learning algorithms;
one or more stored models based on the stored historical data related to the ability of the one or more vehicle operators to reduce impact of the extent of the one or more performance impacting factors.

10. The system of claim 1, further comprising one or more computer-executable instructions configured to determine the anticipated physical and mental health of the one or more vehicle operators while operating the one or more vehicles based on stored historical data related to the physical and mental health of the one or more vehicle operators while operating the one or more vehicles and further optionally based on one or more of the following:
one or more stored insights based on the stored historical data related to the physical and mental health of the one or more vehicle operators while operating the one or more vehicles;
one or more stored configurations;
one or more stored rules based on the stored historical data related to the physical and mental health of the one or more vehicle operators while operating the one or more vehicles wherein the one or more stored rules are further optionally based on one or more self-learning algorithms;
one or more stored models based on the stored historical data related to the physical and mental health of the one or more vehicle operators while operating the one or more vehicles;
one or more inputs provided through one or more graphical user interfaces;
one or more inputs provided without one or more graphical user interfaces.

11. The system of claim 1, further comprising one or more computer-executable instructions configured to check that the assigned one or more segments of the one or more routes and the one or more vehicles to the one or more vehicle operators for the one or more trips comply with the one or more of the following by using one or more stored rules wherein the one or more stored rules are further optionally based on one or more self-learning algorithms:
occupational health and safety related rules;
occupational health and safety related laws;
occupational health and safety related regulations.

12. The system of claim 1, further comprising one or more computer-executable instructions configured to receive and store data related to the one or more assigned trips that the one or more vehicle operators have partially or fully completed.

13. The system of claim 12, further comprising one or more computer-executable instructions configured to check that the received and stored data related to the one or more assigned trips that the one or more vehicle operators have partially or fully completed, comply with the one or more of the following by using one or more stored rules wherein the one or more stored rules are further optionally based on one or more self-learning algorithms:
occupational health and safety related rules;
occupational health and safety related laws;
occupational health and safety related regulations.

14. The system of claim 12, further comprising one or more computer-executable instructions configured to auto-adjust the received and stored data related to the one or more assigned trips that the one or more vehicle operators have partially or fully completed data.

15. The system of claim 14, wherein one or more extents of one or more auto-adjustments are further specific to one or more of the following:
one or more organizations;
one or more fleet types;
one or more target market segments;
one or more driver operator groups;
one or more trip types;
one or more geographic regions;
one or more vehicle models.

16. The system of claim 12, further comprising one or more computer-executable instructions configured to initiate one or more workflows for one or more manual adjustments to the received and saved trips data when one or more stored predefined conditions are satisfied.

17. The system of claim 12, further comprising one or more computer-executable instructions configured to generate one or more of the following related to the received and stored trips data:
one or more insights;
one or more predictions;
one or more recommendations.

18. The system of claim 1, further comprising one or more computer-executable instructions configured to provide one or more workflows allowing interaction through one or more user graphical interfaces for managing one or more life cycles of one or more of the following:
the one or more performance impacting factors;
the one or more anticipated physical and mental health data.

19. A computer-implemented method to tracking and optimizing one or more vehicle operators performance, executed by one or more computing devices each of which comprising of one or more processors coupled to one or more memories, the method comprising assigning one or more segments of one or more routes and one or more vehicles to the one or more vehicle operators for one or more trips based on:
anticipated physical and mental health of the one or more vehicle operators while operating the one or more vehicles during the one or more trips;
extent of one or more performance impacting factors associated with the one or more segments of the one or more routes and ability of the one or more vehicle operators to reduce impact of the extent of the one or more performance impacting factors during the one or more trips.

20. A non-transitory computer readable storage medium with an executable program stored thereon, wherein the executable program is configured to instruct one or more processors to computer-implemented method of claim 19.
